(19) United States
(12) Patent Application Publication
(10) Pub. No.: US 2014/0343389 A1
(43) Pub. Date: Nov. 20, 2014

(54) Wireless Monitoring Device

(71) Applicant: iMobile Healthcare, LLC, West Palm Beach, FL (US)

(72) Inventors: Aaron Goldstein, West Palm Beach, FL (US); Collin Hill, Greenwich, CT (US); William Duckworth, Lake Forest, IL (US)

(73) Assignee: iMobile Healthcare, LLC, West Palm Beach, FL (US)

(21) Appl. No.: 14/282,469

(22) Filed: May 20, 2014

Related U.S. Application Data
(60) Provisional application No. 61/825,173, filed on May 20, 2013.

(51) Int. Cl. A61B 5/00; A61B 5/01

(52) U.S. Cl. CPC A61B 5/0022; A61B 5/01; A61B 5/6832; A61B 5/6804; A61B 5/6803
USPC 600/383; 600/300; 600/549; 600/391; 600/388

(57) ABSTRACT

The present invention relates to a monitor and monitoring system suitable for attachment to the skin of a mammal, including a human. The monitor and monitoring system are designed for continuous wireless real-time measurement of physiological signals and transmission of the measurements to a remote computer or mobile device.

### CROSS REFERENCE TO RELATED APPLICATIONS

This application claims benefit of U.S. Provisional Application No. 61/825,173 filed May 20, 2013 which is incorporated by reference herein in its entirety.

### FIELD OF THE INVENTION

A device and method for continuously monitoring an individual's vital signs.

### BACKGROUND

Health monitoring allows for the discovery and treatment of ailments early in the progression of an illness and helps prevent treatable conditions from becoming life threatening. However, most monitoring is performed intermittently, and if an individual's condition changes rapidly, such changes may not be recognized in time for the appropriate intervention to occur.

For example, chemotherapy patients have depressed immune systems and are at high risk for infection. A fever during chemotherapy treatment is considered a medical emergency (Division of Cancer Prevention and Control, National Center for Chronic Disease Prevention and Health Promotion, Jun. 25, 2013), however effective intervention requires that a patient realize they have a fever before it becomes life threatening. Children who are prone to febrile seizures may need continuous monitoring at home to allow for intervention prior to a seizure occurring. Fevers in elderly, frail, or debilitated individuals are frequently a sign of a severe infection that should be treated immediately (Keating M J III., Klimek J J, Levine D S, et al. Effect of aging on the clinical significance of fever in ambulatory adult patients. J Am Geriatr Soc 1984; 32:282-7). Additionally, vital sign monitoring may be important after surgery, even if a patient has been released from a hospital. For example, after surgery, such as surgery undertaken to restore blood flow to an injured body part, it may be necessary to continuously monitor the area below the injury for temperature and continued circulation.

Traditional thermometers include a liquid that expands or otherwise changes its physical conformation when heated. Thermoresistors in digital thermometers change resistance with changes in temperature which can then be measured and converted to a numerical reading. Aural thermometers use an infrared sensor to measure temperature. However, all of these are designed for intermittent monitoring and require disturbing the patient in order to obtain a reading. There is therefore a need for a means to wirelessly measure temperature and other vital signs on a continuous basis without disturbing the patient.

### SUMMARY

Provided herein is a means for continuously monitoring one or more vital signs in an individual. Further provided herein is a means for wireless, non-invasive continuous monitoring using one or more sensors encased in a sensor housing placed proximate to or against the skin surface of a mammal including a human. In some embodiments, one or more sensors in one or more sensor housings may be placed at one or more locations on the individual. In some embodiments, each sensor may have and/or may transmit a unique ID.

The sensor may be an infrared sensor, thermistor, pulse oximeter, EKG monitor, cardiac telemetry monitor, blood pressure monitor, heart rate monitor, respiration rate monitor, body temperature monitor, and/or an electrocardiogram monitor. In some embodiments, the sensor may transmit a signal to a processor such as a CPU, microprocessor or microcontroller. The sensor measurement is then wirelessly transmitted to a relay unit. The relay unit may track vital sign trends, activate an alarm when pre-determined parameters are exceeded, display the vital sign, and/or transmit the sensor reading to a remote device such as a computer, mobile device or tablet. In some embodiments, the relay unit transmits information to a server via the internet, an intranet, private or public networks and the like to a remote device such as a smart phone or tablet. The server may track vital sign trends, activate an alarm when pre-determined parameters are exceeded, display the vital sign, and/or transmit the sensor reading to a remote device such as a computer, mobile device or tablet. In other embodiments, the relay unit transmits information directly to one or more remote devices. In some embodiments, the remote device may have an application that allows the remote device to track vital sign trends, activate an alarm when pre-determined parameters are exceeded, and/or display the vital sign. In additional embodiments, receipt of sensor readings that exceed pre-set parameters by a remote device may trigger an alarm on the remote device. In another embodiment, sensor readings that exceed pre-set parameters may trigger a message sent to emergency services.

These and other embodiments, features and potential advantages will become apparent with reference to the following description.

### BRIEF DESCRIPTION OF THE DRAWINGS

FIG. 1 is a side view of an embodiment of a sensor unit with a sensor.

FIG. 2 is a view of a sensor unit and an adhesive patch.

FIG. 3 is a view of the inside of an embodiment of a sensor unit.

FIG. 4 is a view of an embodiment of a sensor holder.

FIG. 5 is a view of a system for transmitting vital signs.

FIG. 6 is a view of an embodiment of a relay unit.

FIG. 7 is a view of a system for transmitting a physiological signal to a relay unit for display.

FIG. 8 is a view of an embodiment of a system for transmitting vital signs.

FIG. 9 is a view of an embodiment of a system for transmitting vital signs.

### DETAILED DESCRIPTION

Provided herein is a means for continuous, non-invasive, real-time wireless monitoring of one or more of an individual's vital signs. Further provided herein is a means for sending an alert when an individual's vital signs exceed specific parameters.

The system described herein may be used to continuously monitor vital signs using a system comprising a wireless sensor unit with at least one physiological sensor; a relay unit; and a mobile device. The sensor in the sensor unit may measure physiological signals including, but not limited to, temperature, blood pressure, oxygen levels, electrical conduction, pulse, respiration, heart rate and rhythm and the like. The signal data is then transmitted to the relay unit which transmits the data to the mobile device for access by an interested party. In some embodiments, if the sensor measurement exceeds pre-set parameters, an alarm on either or both the relay device and mobile device may be triggered. In some embodiments, if the sensor measurement exceeds pre-set parameters, emergency services may be notified. The sensor unit may be attached to the body by any means generally used including disposable adhesive patches. In some embodiments, multiple sensor units may be placed around the body with the same or different sensors and the information may be aggregated to provide a more complete picture of an individual's condition. The sensor may have or may transmit a unique id so that information from different sensors on an individual or different sensors on multiple individuals may be distinguished.

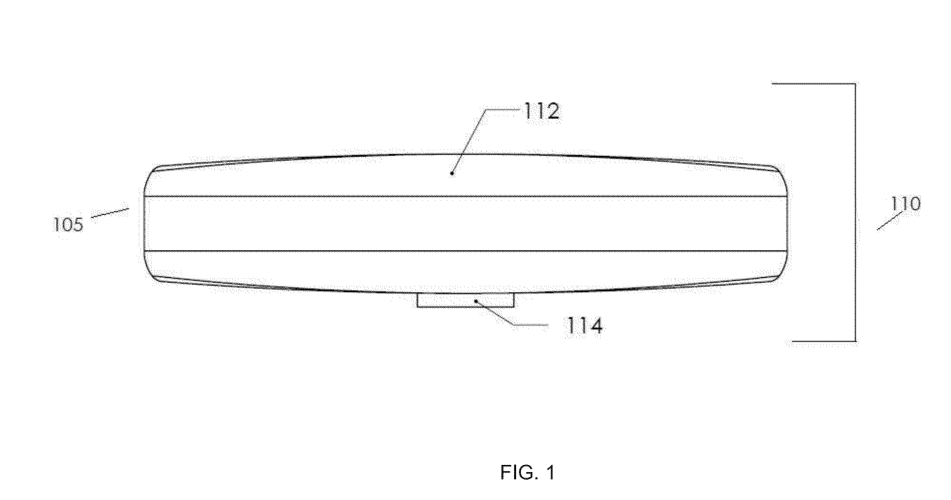

As shown in FIG. 1, a sensor unit 110 comprises a sensor housing 105, a power source and one or more wireless vital sign sensors 114. In some embodiments, the vital sign sensor 114 may be flush with the sensor housing 105. In other embodiments, the vital sign sensor 114 may protrude from a first surface of the sensor housing 105 as shown in FIG. 1. In some embodiments, the sensor unit 110 may be waterproof.

The term “sensor” as used herein refers to any component that is capable of detecting physiological changes through the skin of an individual. Sensors may include any type of electrical, optical, mechanical, and/or chemical non-invasive sensors. The vital sign sensor 114 may be any type of sensor useful in continuous monitoring, including, but not limited to, an infrared sensor, thermistor, pulse oximeter, EKG monitor, cardiac telemetry monitor, blood pressure monitor, heart rate monitor, respiration rate monitor, body temperature monitor, electrocardiogram monitor and the like.

In some embodiments the sensor housing 105 is round as shown in FIG. 1. In other embodiments, the sensor housing is rectangular. In further embodiments the sensor housing is oval. In additional embodiments, the sensor housing is any regular geometric shape.

The sensor may be powered by any means generally used. In some embodiments, the sensor may be powered by an externally accessible battery 112 located on a second surface of the housing 105 of the sensor unit 110. In other embodiments, the sensor may be powered by inductive coupling. In additional embodiments, the sensor may be rechargeable such as through the use of a USB port which plugs into the sensor unit 110, a charging plate, or similar devices.

The sensor housing 105 may additionally encase a replaceable battery, a microprocessor, and a power controller. A wireless continuous monitoring sensor assembly comprises the sensor housing and its components along with a means for attaching the sensor housing to an individual, such as to the core of an individual. In some embodiments, the sensor may be on a first side of the sensor housing and the battery is on a second side of the sensor housing. The sensor data is transmitted to a relay unit which then sends the data to a remote server which sends the data on to a mobile device.

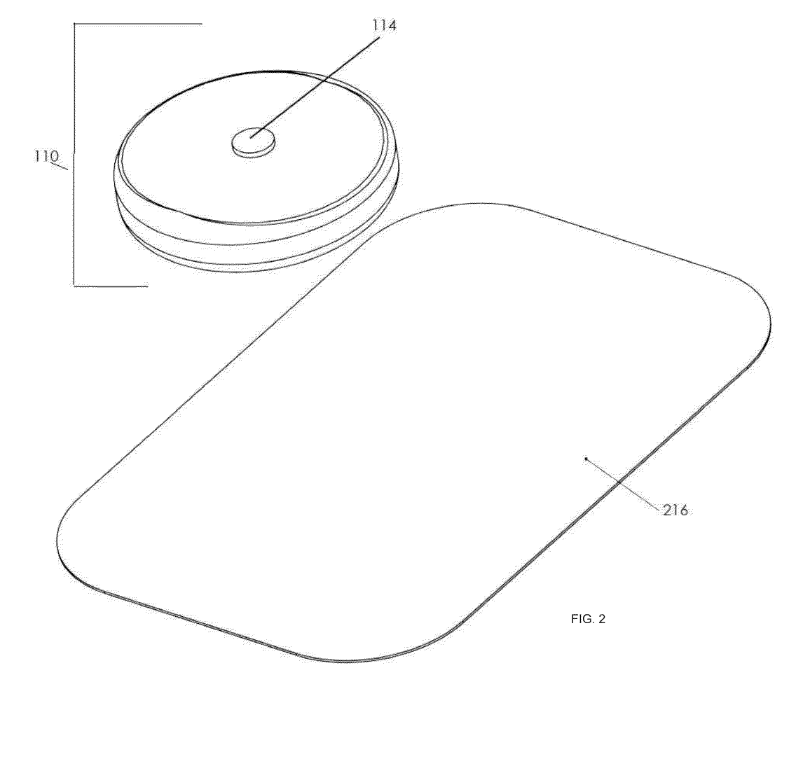

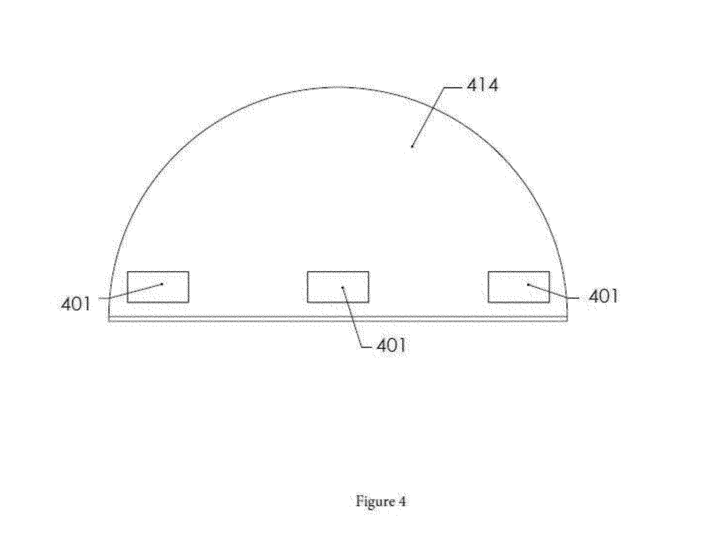

The vital sign sensors described herein are designed to be worn continuously by the individual in need of monitoring. The vital sign sensors may be attached to an individual by any means generally used. In some embodiments, the vital sign sensor may be an epidermal electronic. In other embodiments, the sensor unit is part of a sensor assembly which includes a means for selectively mounting the sensor unit on the skin. In some embodiments, a sensor unit 110 with a vital sign sensor 114 is attached using an adhesive patch 216 such as the one shown in FIG. 2 to form a sensor assembly. In further embodiments, the sensor unit may be worn beneath an item of clothing. In another embodiment, the sensor and/or the sensor unit may be incorporated into wearable jewelry such as a wrist band or chest band. In yet another embodiment, the sensor and/or sensor unit may be incorporated into an item of clothing such as a sock, shirt, pajamas, hat, onesie, or a glove. In some embodiments it may be incorporated into a skull cap as shown in FIG. 4. In some embodiments the means for attaching the sensor housing to the body is disposable. In other embodiments, the means for attaching the sensor housing to the body is reusable.

In some embodiments, the sensor unit 110 may be a one-time-use sensor unit that is provided in a sealed sterile package. In other embodiments, elements of the sensor unit 110 can be disposable while some components are reusable. For example, in some embodiments, the sensor unit 110 may have a replaceable battery 112. In other embodiments, the sensor unit 110 may be rechargeable, for example through a USB port. In a further example, the vital sign sensor 114 may be replaceable. In some embodiments the sensor unit may have an on/off switch. In other embodiments, the sensor unit may automatically turn on when placed in contact with the skin. In additional embodiments, the sensor may be disposable while the sensor housing is not.

The sensor unit may be attached anywhere on the body that is useful in measuring vital signs. In some embodiments, the sensor unit 110 may be selectively attached to the patient's forehead, armpit, arm, chest, foot, abdomen, hand, or back of the ear. In other embodiments, the sensor unit 110 is selectively attached to the body's core (i.e. the body without its arms and legs). In some embodiments, the sensor unit 110 is placed so that the sensor 114 has continuous contact with the skin. In other embodiments, the sensor unit 114 is placed so that the sensor 114 is proximate to the surface of the skin. In some embodiments, multiple sensor units are attached at multiple locations. In additional embodiments, each sensor may have or transmit a unique identification code.

In some embodiments, the sensor unit 110 can include an adhesive backing that helps to facilitate and maintain placement of the sensor by removeably adhering to the patient's skin. In another embodiment, the sensor can comprise adhesive backed foam. The adhesive backing can also help to maintain sensor contact with the user's skin for those sensors that require skin contact. According to some embodiments, conductive sensors may have a conductive gel placed over these sensors.

In some embodiments, the sensor unit may be adhered to the skin using a disposable adhesive patch. The patch may be designed with an adhesive to stay affixed to the skin for 1 or more days, up to 2, 3, 4 or more days. While the patch may be any size, generally the patch is as small as possible yet still provides enough adhesion to hold the sensor in place. In some embodiments the area of the patch is less than about 1 square inch. In other embodiments, the patch may be about 1 inch in diameter. The patch may be circular, oblong or any other regular geometrical shape or irregular shape. In some embodiments, the patch may be colorful and have designs or cartoon pictures.

In some embodiments, the sensors may be part of a patch. In other embodiments, the patch may be placed over the sensor housing as part of a sensor assembly. The patch may be designed with an adhesive to stay affixed to the skin for 1 or more days, up to 2, 3, 4 or more days. While the patch may be any size, generally the patch is as small as possible yet still provides enough adhesion to hold the sensor in place. In some embodiments the area of the patch is less than about 1 square inch. In other embodiments, the patch may be about 1 inch in diameter. The patch may be circular, oblong or any other regular geometrical shape or irregular shape. In some embodiments, the patch may be colorful and have designs or cartoon pictures.

The combination of the patch and sensor preferably have a thickness, ranging from 0.5 mm to about 8 mm, more preferably from about 5 mm to 7 mm, and most preferably about 6.4 mm. The patch preferably includes a body composed of a polymeric material such as a neoprene rubber. In other embodiments, the sensor is part of an epidermal electronic with a thickness of about 1 to about 4 μm.

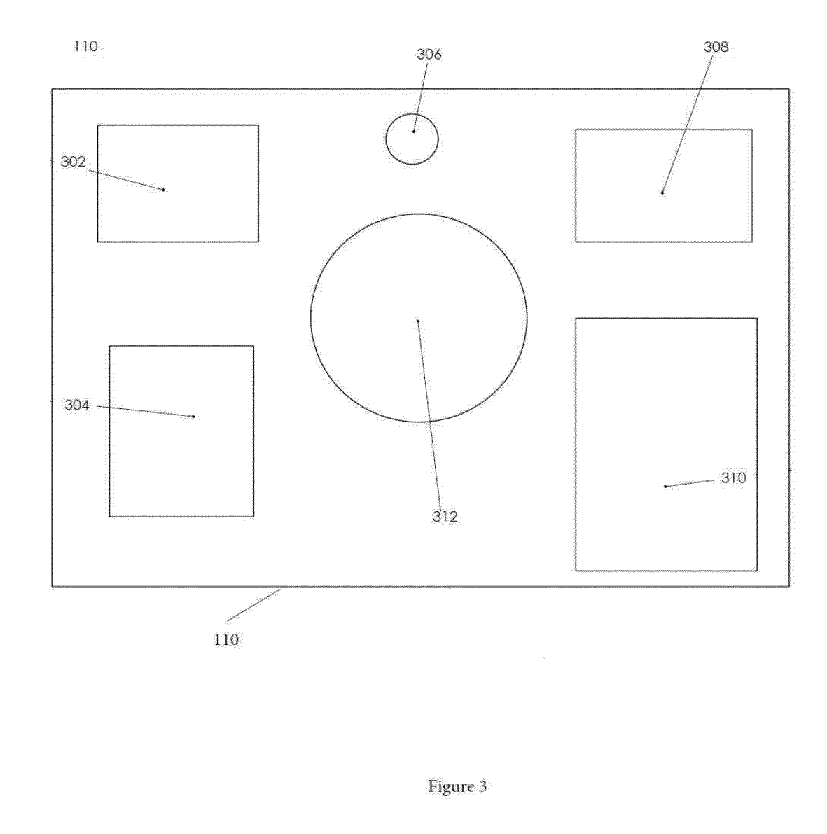

As shown in FIG. 3, the sensor unit 110 may comprise a memory unit 302, an antenna 304, one or more sensors 306, power controller 308, and a CPU 310. In some embodiments, the vital sign sensor 306 may be a thermistor. A thermistor is a temperature-sensing element composed of sintered semiconductor material which exhibits a large change in resistance proportional to a small change in temperature. In some embodiments the thermistor measures core temperature. In other embodiments, the thermistor measures skin temperature. In additional embodiments, the sensor is an infrared sensor, pulse oximeter, EKG monitor, cardiac telemetry monitor, blood pressure monitor, heart rate monitor, respiration rate monitor and the like. In some embodiments, the sensor unit 110 may comprise multiple sensors including 1, 2, 3, 4, 5 or more sensors. Each of the multiple sensors may be the same or different depending on what needs to be monitored in the patient. In some embodiments, a patient may wear more than one sensor unit in more than one place in the body. The sensors in each sensor unit may be the same or different depending on the needs of the patient.

The antenna 304 may transmit a signal by any means generally used. In some embodiments, the antenna 304 may use a wireless protocol. For example, the antenna may use sub-GHZ, ZigBee, Bluetooth, passive RF, or Wi-Fi. In other embodiments, a signal may be sent using infrared or ultrasound wireless control. The antenna length needed for operating at different frequencies is 17.3 cm at 433 MHz, 8.2 cm at 915 MHz, and 3 cm at 2.4 GHz. The 2.4 GHz band has the advantage of enabling one device to serve in all major markets worldwide since the 2.4 GHz band is a global spectrum. However, 433 MHz is a viable alternative to 2.4 GHz for most of the world, and designs based on 868 and 915 MHz radios can serve the US and European markets with a single product. In some embodiments, the frequency may be 14.46 MHz. The antenna may be straight, coiled, or in any configuration useful for transmitting a signal. In some embodiments, the antenna 304 may be replaced with a transceiver. In other embodiments, the sensor signal may be amplified before being transmitted.

The memory unit 302 may be used to store raw measured or processed physiological signals. In some embodiments, the memory unit 302 may store trends in changes in the patient's vital signs. In other embodiments, the memory unit 302 may compare changes in temperature readings to determine the rate at which an individual's temperature is increasing. In additional embodiments, the memory unit 302 may store data until a vital sign exceeds certain parameters at which point the sensor unit sends a signal to a relay unit which may analyze the information or transfer the information directly to a computer or mobile device for analysis.

In some embodiments, the CPU 310 takes in raw voltage or resistance data from the thermistor, converts it into useable temperature data and then provides new binary temperature data to be transmitted to the relay unit.

In some embodiments, the memory unit 302 and the CPU 310 may be replaced by a microcontroller. The microcontroller may include a CPU storage/memory (e.g., RAM, ROM, EEPROM, flash), general purpose input/output (GPIO), analog-to-digital (A/D) and digital-to-analog (D/A) converters, as well as digital signal processors (DSP).

In some embodiments, the power controller 308 may be an intelligent selection of transmitter power output in a communication system to achieve good performance within the system. In some embodiments, the power controller 308 may be a proportional-derivative-integrative controller.

The power source 312 may be a permanent, replaceable or rechargeable battery. In some embodiments, the sensor unit 110 may be recharged using a USB port or other similar device. In additional embodiments, the power source 312 may be rechargeable using a charging plate. In additional embodiments, the power source may be replaced by the patient as needed.

FIG. 4 depicts an additional embodiment of a sensor unit. As shown in FIG. 4, a skull cap 414 houses one or more vital sign sensors 401. The sensors may be any type of vital sign sensor useful in continuous monitoring, including, but not limited to, an infrared sensor, thermistor, pulse oximeter, EKG monitor, cardiac telemetry monitor, blood pressure monitor, heart rate monitor, respiration rate monitor, and the like. Vital sign sensors 401 may be the same or different. In some embodiments, the vital sign sensors 401 are equally spaced on the skull cap 414 as shown. In other embodiments, a single vital sign sensor 401 is placed in the skull cap 414. In additional embodiments, a plurality of vital sign sensors 401 may be placed as needed throughout the skull cap.

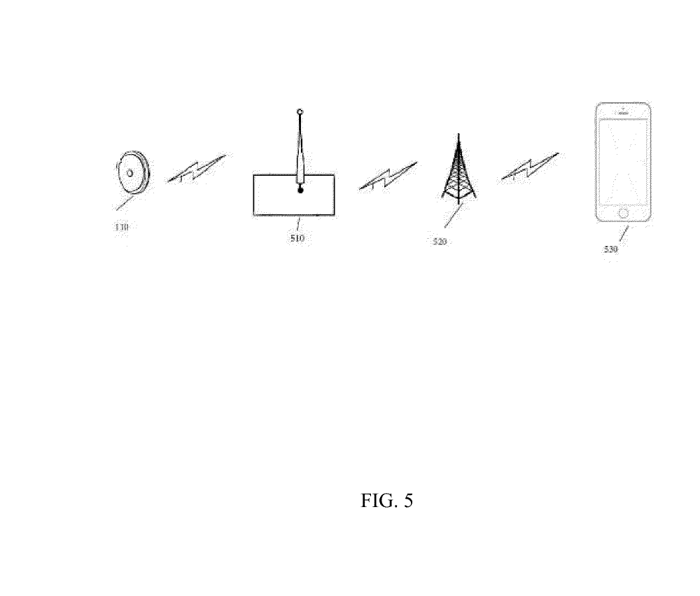

As shown in FIG. 5, the readings from the sensor unit 110 are sent to a nearby relay unit 510. Readings may be sent by any means generally used including, but not limited to, sub-GHZ, ZigBee, Bluetooth, passive RF, or Wi-Fi. In other embodiments, a signal may be sent using infrared or ultrasound wireless control. In some embodiments, the relay unit 510 may record multiple sensor readings. In other embodiments, the relay unit 510 may determine trend lines based on sensor readings. In some embodiments, the relay unit 510 may display the sensor measurements on a first surface of the relay unit. In other embodiments, the relay unit 510 merely transmits the information to a remote device 530 such as a computer, tablet, and mobile device such as a smart phone or similar devices via telecommunication. The relay unit 510 may send a signal to a cellular network 520 using Wi-Fi, SMS, WLAN, or a similar communication protocol. In some embodiments, the relay unit 510 may send a signal to a remote server. In some embodiments, the server may be part of a private network. In another embodiment, the signal may be sent via the internet. In further embodiments, it may be sent over a secured line. In additional embodiments, the signal may be encrypted. The relevant information from the sensor unit 110 is then sent from the server to the remote device. The information may be displayed by any means generally used. In some embodiments, the information is sent via a text message. In another embodiment, the information is sent to an application on the smart device. In a further embodiment, the information is sent via a pre-recorded message. The server may send as little or as much information as desired. In some embodiments, the server only sends information to the remote device when certain parameters are exceeded. In other embodiments, the server may continually update the remote device. In additional embodiments, the remote device may be periodically updated. In some embodiments, the information from the sensor may trigger an alarm in the relay device and/or remote device when the data exceeds certain parameters. For example, if temperature is being measured, an alarm may trigger if the sensor measures a temperature below 95° F. or above about 100° F. In the case of a child, an alarm may be triggered if the temperature of the child reaches 102° F. In another embodiment, an alarm may be triggered if particular trends are noted even if the sensor is not measuring a temperature exceeding pre-set parameters. For example, if an individual's temperature is trending upwards over a certain period of time, an alarm may be triggered. In additional embodiments, a processor in the relay unit and/or in an application in the remote device may compensate for normal fluctuations in vital signs. For example, body temperature normally fluctuates by almost a degree Fahrenheit during the course of the day with the body temperature lower in the morning and higher in the evening. Therefore, a slight upwards trend in temperature during the course of the day may not trigger any sort of alarm in the remote device. In other embodiments, an alarm may be triggered if a particular trend is observed. In some embodiments, if the sensor reaches a particular threshold, emergency services or a doctor's office may be contacted. Parameters for an alarm to be triggered may be pre-set by the manufacturer or may be set by the individual monitoring the sensor wearer. In some embodiments, the server and/or remote device may store historical data from the sensor(s) allowing production of the sensor data for a physician. In some embodiments, information from the sensors may be available through a web portal.

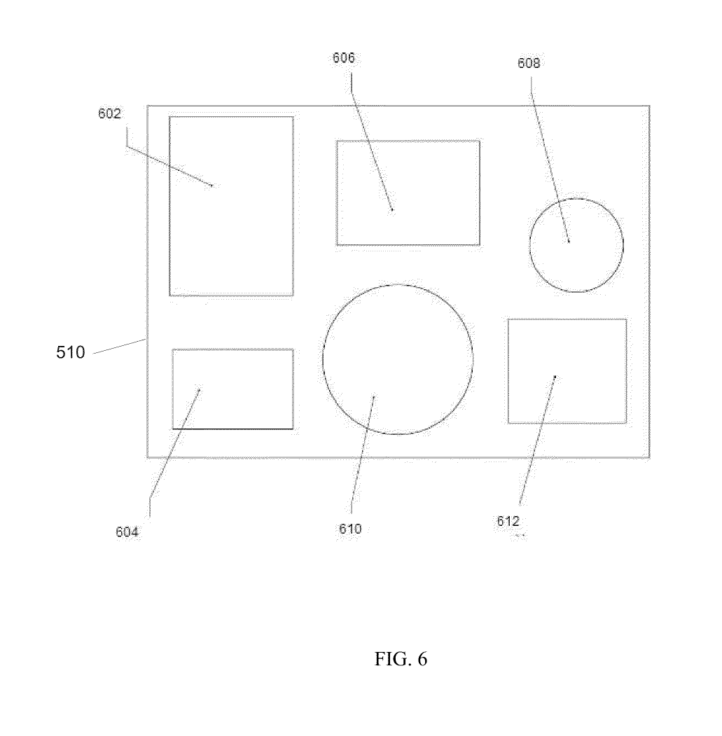

As shown in FIG. 6, the relay unit 510 may comprise a CPU 604, a transceiver 612, a power controller 602, and a Wi-Fi radio 606. In some embodiments, the relay unit 510 may plug into a wall to obtain power. In other embodiments it may use a replaceable battery 610. In further embodiments it may be rechargeable, for example through a USB port.

The CPU 604 is the component controlling other components in the relay unit 510. In some embodiments, it may analyze the data from the sensor. In general, the more speed and data analysis required, the more power is needed. Therefore a sleep function is often used in order to save power. At certain times or if certain events happen, the CPU wakes up, makes the necessary calculations, communicates with relevant components and returns to sleep mode.

The power controller 602 selects transmitter power output to achieve good performance within the communication system. The transceiver 612 may be any type of transceiver generally used. In some embodiments it may comprise a radio with an antenna. The Wi-Fi radio 606 takes the signal received and sends it to a remote server.

The relay unit 510 may additionally comprise a status light 608. Such a status light may change colors when charging, when turned on, when a signal is being sent, when a signal is being transmitted, or any additional status desired. The status light may convey information by remaining steady, blinking, blinking in a particular pattern, displaying a particular color, turning off or any other means status lights convey information.

In some embodiments, the relay unit 510 may additionally store information received from the sensor. In other embodiments, the relay unit 510 may analyze the information received from the sensor and compare it to previous readings to determine if there is a trend in the vital sign, particularly a trend indicating there is an issue.

In some embodiments, the sensor unit 110 may be remotely activated. For example, it may be less necessary for vital signs to be monitored while a patient is awake or if the patient is hospitalized. The sensor may therefore be programmed by the relay unit 510 or the remote device 520 to turn on or off at certain times of day. In other embodiments, the sensor unit 110 may be switched on or off remotely and/or manually.

In some embodiments, the vital signs of an individual are monitored continuously during real time by attaching a sensor in a sensor unit to an individual, measuring the vital signs and sending the vital signs to a relay unit. The data from a physiological signal may be sent to the relay unit using sub-GHZ, ZigBee, Bluetooth, passive RF, or Wi-Fi. In other embodiments, a signal may be sent using infrared or ultrasound wireless control. In some embodiments, the relay unit may record and analyze the information received from the sensor. The information is then sent from the relay unit to a mobile device which may sound an alarm if a pre-determined parameter is exceeded. In other embodiments, the relay unit and/or mobile device may contact emergency services if the pre-determined parameter is exceeded.

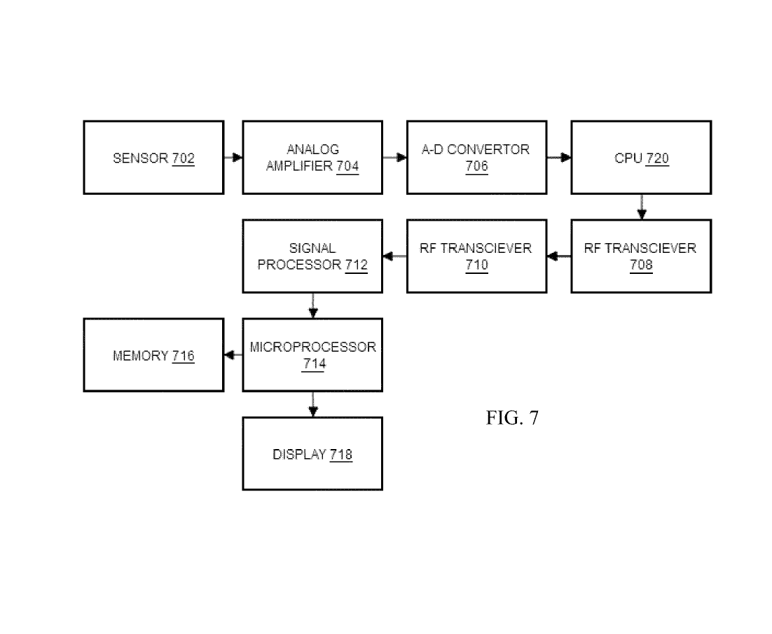

As shown in FIG. 7, in some embodiments a physiological signal is measured by a sensor 702. The signal from the sensor 702 is amplified by an analog amplifier 704. The analog signal from the analog amplifier 704 is converted to a digital signal by an A-D convertor 706. The digital signal is then sent to a CPU 720 for processing. After processing, the signal is sent to a first RF transceiver 708 and then to a second transceiver 710 in a relay unit. The signal from the second RF Transceiver 710 is then processed by a signal processor 712 to optimize it and the resulting vital sign measurement is analyzed by the microprocessor 714, stored in the memory 716 and displayed 718 on a first side of the relay unit.

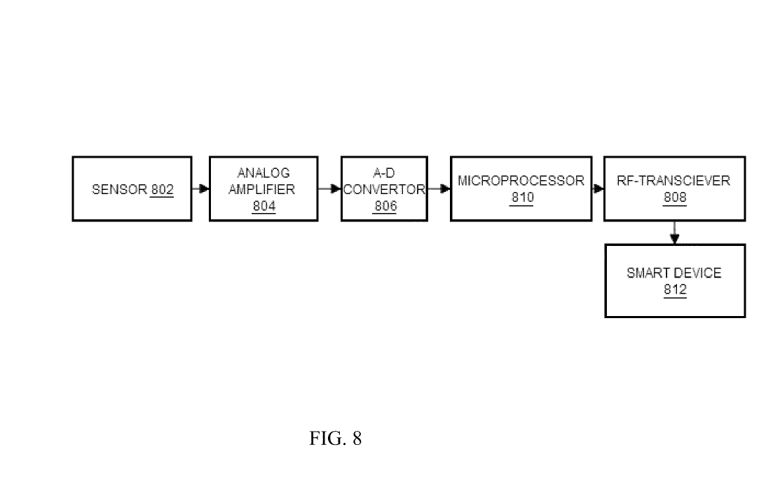

As shown in FIG. 8, in some embodiments the sensor unit sends the signal directly to a smart device 812 such as a computer, tablet or smart phone and does not transmit a signal to a relay unit. The signal may be sent to the smart device 812 via Bluetooth, Wi-Fi, SMS, WLAN, or a similar communication protocol. In some embodiments, the sensor unit may send a signal to a server in the cloud which then transmits a signal to a smart device 812.

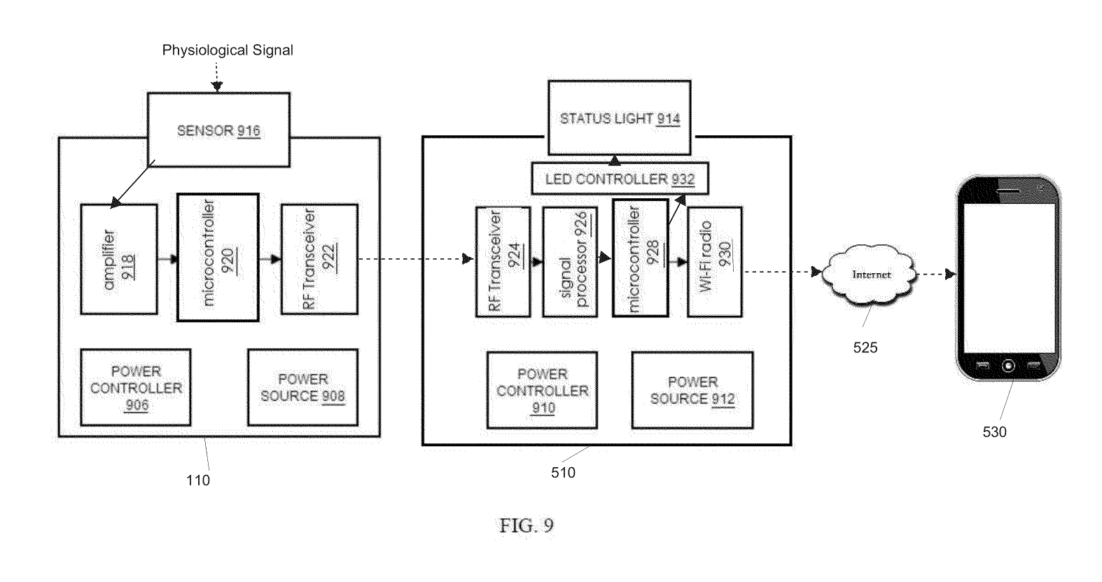

In other embodiments, as shown in FIG. 9, a sensor 916 in a sensor unit 110 measures a physiological signal. The sensor 916 sends the signal to an amplifier 918. The amplifier 918 transmits the signal to the microcontroller 920. The microcontroller 920 converts the signal from analog to a digital signal, processes the signal, records the signal and transmits it to the RF transceiver 922. The RF transceiver 922 sends the signal to a second RF transceiver 924 in the relay unit 510. The RF transceiver 924 then sends the signal to a signal processor 926 which improves the accuracy and reliability of the signal. The cleaned signal is then sent to a microprocessor 928 where the digital signal is converted to a vital sign measurement and recorded.

In some embodiments, the relay unit 510 may include a status light 914, generally an LED light. The microcontroller 928 sends a signal to the LED controller 932 to alter the status light as appropriate. Such a status light may change colors when charging, when turned on, when a signal is being sent, when a signal is being transmitted, or any additional status desired. The status light may convey information by remaining steady, blinking, blinking in a particular pattern, displaying a particular color, turning off or any other means status lights convey information.

The vital sign measurement is then sent to Wi-Fi radio 930 or transceiver which transmits the measurement to a remote server 525 via Wi-Fi or other similar communication method. For example, the relay unit may send a signal to a remote server using Wi-Fi, SMS, WLAN, or a similar communication protocol. In some embodiments, the server may be part of a private network. In another embodiment, the signal may be sent via the internet. In further embodiments, the signal may be sent over a secured line. In additional embodiments, the signal may be encrypted. The remote server then sends the vital sign measurement and/or any trends in measurements and other desired data to a remote device 530 such as a computer, tablet, mobile device such as a smart phone, or similar device.

The relevant information from the sensor is then displayed on the screen of the remote device with or without trend analysis. In some embodiments, the information from the sensor may trigger an alarm in the remote device when the data exceeds certain parameters. For example, if temperature is being measured, an alarm may trigger if the sensor measures a temperature below 95° F. or above about 100° F. In the case of a child, an alarm may be triggered if the temperature of the child reaches 102° F. For example, if an individual's temperature is trending upwards over a certain period of time, an alarm may be triggered earlier. In additional embodiments, a processor in the relay unit and/or in an application in the remote device may compensate for normal fluctuations in vital signs. For example, body temperature normally fluctuates by almost a degree Fahrenheit during the course of the day with the body temperature lower in the morning and higher in the evening. Therefore, a slight upwards trend in temperature during the course of the day may not trigger any sort of alarm in the remote device. In other embodiments, an alarm may be triggered if a particular trend is observed. In some embodiments, if the sensor reaches a particular threshold, emergency services or other parties may be contacted.

Those having skill in the art will appreciate that there are various logic implementations by which processes and/or systems described herein can be effected (e.g., hardware, software, and/or firmware), and that the preferred vehicle will vary with the context in which the processes are deployed. “Software” refers to logic that may be readily readapted to different purposes (e.g. read/write volatile or nonvolatile memory or media). “Firmware” refers to logic embodied as read-only memories and/or media. “Hardware” refers to logic embodied as analog and/or digital circuits. If an implementer determines that speed and accuracy are paramount, the implementer may opt for a hardware and/or firmware vehicle; alternatively, if flexibility is paramount, the implementer may opt for a solely software implementation; or, yet again alternatively, the implementer may opt for some combination of hardware, software, and/or firmware. Hence, there are several possible vehicles by which the processes described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations may involve optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood as notorious by those within the art that each function and/or operation within such block diagrams, flowcharts, or examples can be implemented, individually and/or collectively, by a wide range of hardware, software, firmware, or virtually any combination thereof. Several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in standard integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies equally regardless of the particular type of signal bearing media used to actually carry out the distribution. Examples of a signal bearing media include, but are not limited to, the following: recordable type media such as floppy disks, hard disk drives, CD ROMs, digital tape, and computer memory.

In a general sense, those skilled in the art will recognize that the various aspects described herein which can be implemented, individually and/or collectively, by a wide range of hardware, software, firmware, or any combination thereof can be viewed as being composed of various types of “circuitry.” Consequently, as used herein “circuitry” includes, but is not limited to, electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), circuitry forming a memory device (e.g., forms of random access memory), and/or circuitry forming a communications device (e.g., a modem, communications switch, or optical-electrical equipment).

Those skilled in the art will recognize that it is common within the art to describe devices and/or processes in the fashion set forth herein, and thereafter use standard engineering practices to integrate such described devices and/or processes into larger systems. That is, at least a portion of the devices and/or processes described herein can be integrated into a network processing system via a reasonable amount of experimentation.

The foregoing described aspects depict different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality.

We claim:

1. A system for continuously monitoring vital signs in an individual comprising:
a wireless sensor unit with at least one physiological sensor;
a relay unit; and
a mobile device;
wherein the sensor measures a physiological signal, transmits a signal containing the measurement to the relay unit; and
wherein the relay unit transmits the measurement to the mobile device.

2. The system of claim 1, wherein the sensor is a thermistor.

3. The system of claim 1, wherein if the sensor measurement exceeds pre-set parameters, an alarm is triggered on the mobile device.

4. The system of claim 1, further comprising a means for releasably attaching the sensor unit to the body of the individual with a disposable means of attachment.

5. The system of claim 4, wherein the means for attaching the sensor unit is an adhesive patch.

6. The system of claim 4, wherein the means for attaching the sensor unit to the body is a skull cap.

7. The system of claim 1, wherein the system comprises multiple wireless sensor units with at least one physiological sensor.

8. The system of claim 1, wherein the relay unit displays the measurement on a first surface of the relay unit.

9. The system of claim 1, wherein the physiological sensor transmits a unique id.

10. The system of claim 1, wherein if the sensor measurement exceeds pre-set parameters, emergency services are notified.

11. A wireless continuous monitoring sensor assembly comprising:
a sensor housing;
a thermistor;
a replaceable battery;
a microprocessor;
a power controller;
a means for selectively mounting the sensor housing on a core of a body; and
a means for transmitting sensor data to a relay unit;
wherein the thermistor extends from a first side of the sensor housing such that the thermistor will be in contact with a core of a body of an individual while the microprocessor and power controller are maintained within the housing; and wherein the replaceable battery is accessible from a second side of the sensor housing such that it does not interfere with the positioning of the thermistor.

12. The wireless continuous monitoring sensor assembly of claim 11, wherein the means for selectively mounting the sensor housing on the core of the body is an adhesive patch.

13. The wireless continuous monitoring sensor assembly of claim 11, wherein the means for selectively mounting the sensor housing on the core of the body is a shirt.

14. The wireless continuous monitoring sensor assembly of claim 11, wherein the relay unit comprises a means for sending data from the thermistor to a remote server.

15. The wireless continuous monitoring sensor assembly of claim 14, wherein the remote server transmits data from the thermistor to a mobile device.

16. The wireless continuous monitoring sensor assembly of claim 11, wherein the relay unit comprises a means for sending data from the thermistor to a mobile device.

17. A method for wireless continuous real-time monitoring of a mammal's vital signs comprising:
attaching a sensor to a body core of a mammal;
measuring the vital signs of the mammal;
wirelessly transmitting the vital signs to a relay unit;
recording the vital signs in the relay unit; and
transmitting the vital sign measurements to a mobile device;
wherein the mobile device sounds an alarm if the vital sign measurement exceeds a pre-determined parameter.

18. The method of claim 17, wherein the sensor is in a sensor unit.

19. The method of claim 17, wherein the sensor is a thermistor.

20. The method of claim 17, wherein the relay unit contacts emergency services if the vital sign measurements exceed a pre-determined parameter.
